(19) United States
(12) Patent Application Publication
(10) Pub. No.: US 2013/0030255 A1
(43) Pub. Date: Jan. 31, 2013

(54) BIOCOMPATIBLE IMPLANT DEVICE

(71) Applicant: William Ben Embry, II, Malden, MO (US)

(72) Inventor: William Ben Embry, II, Malden, MO (US)

(21) Appl. No.: 13/555,230

(22) Filed: Jul. 23, 2012

Related U.S. Application Data
(60) Provisional application No. 61/511,725, filed on Jul. 26, 2011.

(51) Int. Cl. A61B 5/00; A61M 5/00

(52) U.S. Cl. 600/300; 604/131

(57) ABSTRACT

A biocompatible implant device (BCI) including a battery, a processor and at least one customizable operations module. The BCI is preferably rechargeable wirelessly via induction, and thus also contains an induction module including an induction coil connected to the battery. The BCI further includes a transceiver for communicating with external electronic devices. These features allow the BCI to be fully implanted with minimal biological issues, and without the need to remove the BCI to replace batteries, control the device or to obtain information therefrom.

### CROSS-REFERENCE TO RELATED APPLICATION

The present application claims priority to and incorporates herein by reference U.S. Provisional Patent Application Ser. No. 61/511,725 filed on Jul. 26, 2011.

### FIELD OF THE INVENTION

The present invention relates to a biocompatible implant device and, more particularly, to electronic devices which can be implanted into living tissues for long durations with minimal adverse reactions, and which can be wirelessly recharged in vivo (inside living tissue) without additional trauma to the surrounding living tissue.

### BACKGROUND OF THE INVENTION

Many limitations and complications have surrounded the field of biocompatible implantable devices (BCI devices). Primarily, a conventional power source (i.e., battery) will not last long enough to be able to maintain a long-term powered functional implant with the numerous capabilities of the aforementioned advanced biocompatible implantable device. Few methods for recharging a power source are readily or commercially available for implanted devices inside of living tissues that are safe and/or effective. Thus, most implantable devices generally cannot maintain a functional environment while implanted inside of living tissues for extended periods of time.

Additionally, for many necessary tasks, such an implantable device requires an integrated computer system with wireless capabilities and interchangeable third party electronics. Maintaining a power source strong enough to run such hardware and any needed software or operating system(s) and transceivers for any long duration are impractical with conventional power sources inside living tissue. Further, such an implantable device would need to be biocompatible, as many materials used in implantable devices have been found to cause extreme tissue reactions and some are even carcinogenic.

Solving such problems would open the door in the advanced biocompatible implantable device field to enable an entirely new possibility of functions/applications/interactions between living tissue, advanced powered rechargeable integrated computer BCI devices, and external apparatuses/technologies/networks.

### SUMMARY OF THE INVENTION

The aforementioned complications/problems can all be resolved by an implant which maintains an integrated computer system with connection ports for additional devices such as GPS location devices, Radio Frequency Identification devices, medical research/monitoring devices, alternative secondary power source recharging devices, and so forth. The implant hosts a built in power source (e.g., a battery) with recharging capabilities via electromagnetic induction, or a self-sustaining power source (e.g., a thermoelectric generator). As will be understood, thermoelectric generators convert heat (e.g., from a body) into electrical energy, and thereby are able to “recharge” by generating additional energy to be stored by an internal battery and/or used by the device. The implant also has the ability to be accessed remotely, communicate wirelessly, and can be turned on/off remotely or based on previous instructions. Software and firmware may be updatable wirelessly. The ability to turn on/off the integrated computer, chronic frequency transmission, and integrated electromagnetic induction recharging magnetic field greatly decreases the chance for serious tissue reactions. It also allows for low-power consumption and power source preservation/longevity.

The implant is preferably made of biocompatible materials so as to greatly reduce the chance for adverse tissue reactions including, but not limited to, cellular necrosis, neoplasia (tumors/cancers), inflammation, soft tissue swelling, immune disorders, tissue rejection, or other tissue reactions. Some non-limiting examples of biocompatible components and materials are: anti-humidity gases, titanium/titanium allow, polyurethane, modified silicon semiconductors, soda lime, silicon, cobalt, etc. The implant is preferably hermetically sealed to prevent any moisture or fluid from entering the implant, although some constructions/embodiments may require a non-hermetically sealed design needed for monitoring/testing surrounding tissues. In a hermetically sealed environment, the infrastructure of the BCI device can maintain its integrated components (circuitry, processor, power source, electrical connections, antennae, transmitter, etc.) in a functional environment inside of living tissue by preventing fluid/moisture infiltration. As technology advances are made, the BCI device can even easily integrate safer/more efficient self-sustaining power sources (i.e. solar power, thermoelectric generators, small reactors, chemical, and so forth.)

Such an advanced biocompatible implantable device may also serve as a bridge between living tissue and other advanced biocompatible implantable devices, external apparatuses or technologies. Adding additional devices to connection ports of a BCI will provide interchangeable functionality. Such additional devices may include, but are not limited to, devices for living tissue that can be utilized for the monitoring/research of living tissues (oncology research, pathology research, organ/tissue research, medicinal research), controlled medicinal delivery at intervals controlled/monitored remotely, monitoring hormonal or chemical levels in the body, long term plant implantation research, networking/satellite/OWT transceivers, biocompatible self-sustaining power apparatuses (e.g., thermoelectric generators), passive identification devices, advanced computer chip/microchip additions, location/tracking service devices, and so forth.

The present advanced biocompatible implantable device can be safely and easily recharged by a small, portable apparatus which could be worn in an adjustable strap/harness over the implanted device. Such a portable apparatus may recharge the BCI via electromagnetic induction, and may have the ability to initiate/terminate the BCI's recharging functions and monitor/display power source life via external controls and an external display. Thus, the BCI device provides for great flexibility. The BCI device may maintain an active wireless bridge to safely communicate and cross the gap between living tissue and external technology, and the device's application capabilities can handle a wide variety of tasks/files/data/functions and also provide a functional bridge for other networks/devices/applications.

Further, the BCI device can preferably remain implanted and function for extended periods of time without causing interferences with the physiological functions of tissues or without causing abnormal or harmful tissue reactions (i.e. excessive granulation tissue, acute/chronic inflammation, neoplasia, foreign body draining tract lesions, systemic/local toxicity, and so forth.) This is achieved through a number of integrated safety features, including the ability to automatically turn on/off various components within the device as needed. Such components may also be turned off manually via an integrated OWT/Satellite transceiver (discussed below). This feature also maintains the longevity of the integrated power source to limit the need for constant recharging, thus reducing the amount of side effects/reactions to living tissue via magnetic fields/phenomena. The external components, as well as most internal components, of the BCI device are preferably biocompatible and specially designed to also minimize tissue reactions.

The external components are preferably biocompatible and non-porous to facilitate a functional environment internally, or may be altered with one-way and/or two-way ports to allow for integrated monitoring/delivery/collection systems. It is understood that other embodiments may be utilized and certain alterations made without departing from the scope of the invention, and therefore the monitor/delivery of specific chemicals, elements, hormones and materials will not be discussed in great detail in this description. Various sizes of the BCI device can be produced depending on functional desire/tissue type, with smaller versions for living tissue being ideally less than approximately 20-30 mm in length, 7-10 mm in height, and 10-15 mm in width.

While the present BCI applications have been most clearly described in the context of living tissues (human, animal, plant), similar systems may be easily modified for use in inanimate objects. For example, a BCI device may be easily implanted into inanimate objects for long-term communication/research/tracking uses. Other possible uses include, but are not limited to, living tissue/organ research in the medical/veterinary fields, tracking living entities for prolonged periods of time for research and storing/analyzing/transmitting the data to an external apparatus via a network (migratory patterns of isolated animals), advanced location tracking of high profile or valuable entities (living/inanimate), satellite/“open wireless technology” (e.g., Bluetooth) control of advanced BCI device functions/applications, advance integrated enhancement of entities (living/inanimate), controlled/measured administration/monitoring/tracking of drugs (chemotherapy, hormones, and so forth).

It is further understood that various tissues will accept BCI devices differently and that the preferred embodiment illustrated/described may require various alterations to facilitate tissue acceptance. These alterations may include, but are not limited to, varying external texture/composition, adding tissue acceptance enhancement bio-layers/gels/chemicals, added external bio-scaffolding layers facilitating tissue uptake via spurring new, healthy tissue growth enveloping the BCI device, the addition of a biocompatible dissolvable non-reactive bio-layer thereby delaying/minimizing tissue reactions/rejections to the BCI device while increasing tissue/implant adjustment periods, varying internal component's composition/materials, and so forth.

Preferred method of implantation may include, but is not limited to, surgical (subcutaneous tissues/organs, intramuscular, etc.) or injections (subcutaneous tissues/organs, intramuscular, ultrasound guided, etc.).

Accordingly, it is to be understood that the embodiments of the invention herein described are merely illustrative of the applications of the principles of the invention. It should also be made apparent that an invention having significant advantages over present biocompatible implants has been provided. While the invention is shown in only a few of its forms, it is not so just limited but is susceptible to various changes and modifications as more advanced technologies, software, etc. become readily available. Reference herein to details of the illustrated embodiments is not intended to limit the scope of the claims, which themselves recite those features regarded as essential to the invention. It is also evident that this invention will be able to withstand and integrate technological advances unforeseen presently and can be adapted to suit a wide range of networks/functions. The self-contained BCI device contains the design to be readily updated/altered accordingly with future technological advances.

Other objects, features, and advantages of the present invention will become apparent with reference to the drawings and detailed description that follow.

### DESCRIPTION OF THE DRAWINGS

FIG. 1A is a top plan view of one embodiment of a biocompatible implant device (“BCI”) constructed according to the teachings of the present invention.

FIG. 1B is a front elevation view of the BCI of FIG. 1A.

FIG. 1C is a side elevation view of the BCI of FIG. 1A.

FIG. 2A is a side cross-sectional view of the top section of a BCI constructed according to the teachings of the present invention.

FIG. 2B is a side cross-sectional view of the bottom section of a BCI constructed according to the teachings of the present invention.

FIG. 3A is a top cross-sectional view of the top section of a BCI constructed according to the teachings of the present invention.

FIG. 3B is a bottom cross-sectional view of the bottom section of a BCI constructed according to the teachings of the present invention.

FIG. 4 is a block diagram of an exemplary apparatus for charging and communicating with a BCI according to the teachings of the present invention.

FIG. 5 is a perspective view of the apparatus charging and communication with a BCI of FIG. 4 as installed in a wearable band.

FIG. 6 is a block diagram of possible communications between a BCI and external devices.

It should be understood that the present drawings are not necessarily to scale and that the embodiments disclosed herein are sometimes illustrated by fragmentary views. In certain instances, details which are not necessary for an understanding of the present invention or which render other details difficult to perceive may have been omitted. It should also be understood that the invention is not necessarily limited to the particular embodiments illustrated herein. Like numbers utilized throughout the various figures designate like or similar parts or structures.

### DETAILED DESCRIPTION

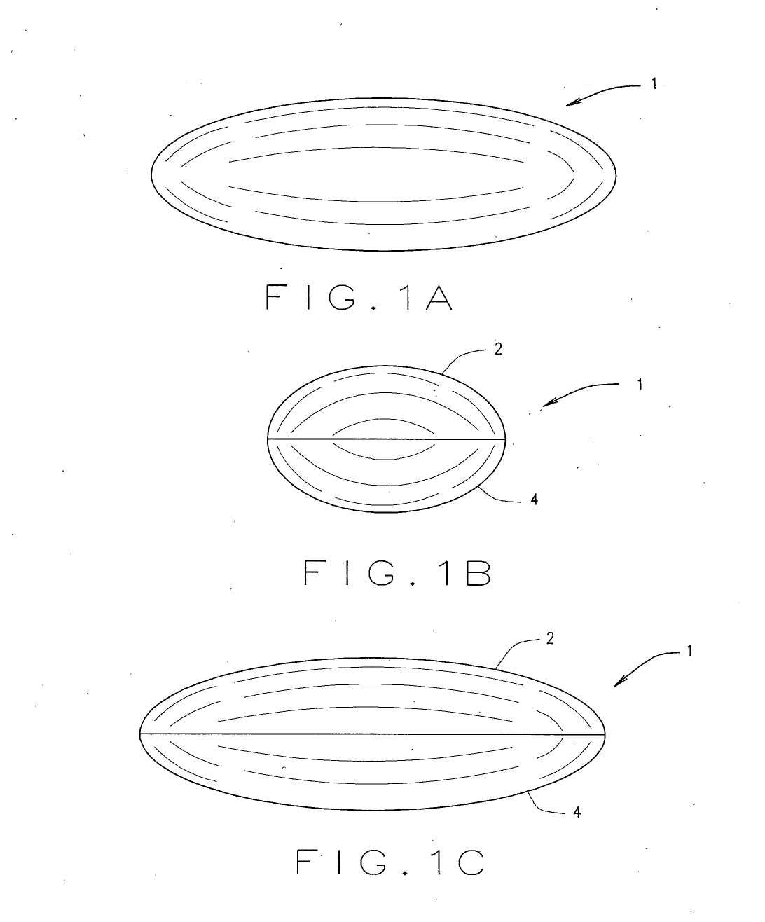

Referring now to the drawings and, more particularly, to FIGS. 1A-1C, a biocompatible implant device (“BCI”) 1 is shown. As can be seen, BCI 1 includes a top hemisphere 2 and a bottom hemisphere 4. Top hemisphere 2 and bottom hemisphere 4 may be hermetically sealed together in an airtight seal to prevent anything from entering or exiting the BCI 1 upon implantation. Top hemisphere 2 and bottom hemisphere 4 are preferably formed of known biocompatible materials to reduce the chance of adverse tissue reactions. As shown, top hemisphere 2 and bottom hemisphere 4 fit together to form a generally rounded, oblong shape.

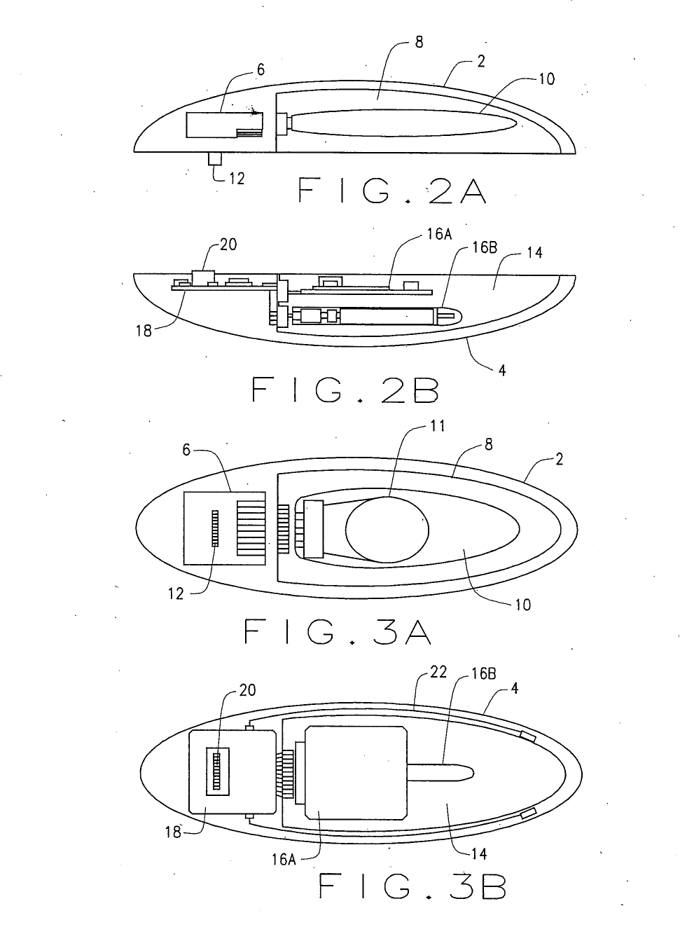

FIGS. 2A and 2B illustrate a side cross-sectional view of the top and bottom hemispheres 2, 4 of an example BCI 1. As seen in FIG. 2A, the top hemisphere 2 may include a power source, such as rechargeable battery 6 for storing electrical power. Top hemisphere 2 may also include a cavity 8 in which an induction module 10 may reside. Induction module 10 may include an electromagnetic induction coil (shown as part of induction module 10 in FIG. 3A, discussed below). Induction module 10 is preferably in electrical communication with battery 6 for charging the battery 6. Battery 6 is preferably also electrically connected to a male power connection port 12.

As is shown in FIG. 2B, bottom hemisphere 4 preferably also includes a cavity 14 in which various components may reside. As shown, cavity 14 contains two example modules 16A and 16B, which are electronically connected to a processor 18 via connection ports. As will be understood, BCI 1 may be sized to house more or less than two customizable modules 16A, 16B to perform various functions. Processor 18 is preferably electrically connected to a female power connection port 20 for interfacing with the male power connection port 12 of the top hemisphere 1 so as to provide electrical power from battery 6 to the processor 18 and modules 16A, 16B. Modules 16A and 16B may perform any desirable function, but as shown in FIG. 2B, module 16A is a GPS chip which utilizes processor 18 and wireless transceiver 22 (discussed below) for receiving GPS signals to locate/track the BCI device 1. Transceiver 22, shown in FIG. 3B, receives data from and transmits data to sources outside the BCI 1 (and outside the body into which it is implanted). As a non-limiting example, Module 16B may be an RFID device for passive identification, but could be any other type of device as needed. Alternatively, modules 16A and 16B may work together to perform various functions. As will be understood, processor 18 is a built-in processor for performing functions within BCI 1, such as updating/storing files and executing operations and applications, and may include memory and other necessary features. Processor 18 and transceiver 22 may work in conjunction for external technological communication via OWT/Satellite.

In operation, such an exemplary BCI 1 may be implanted into the body of a person/animal/object, and the battery 6 may be wirelessly charged and recharged via electromagnetic induction via the induction module 10. Once implanted, processor 18 may draw power as needed from battery 6. By way of example, operations module 16A may then track the location of the BCI 1 (and thereby of the person/animal/object into which the BCI 1 is implanted). Wireless transceiver 22 and/or operations module 16A may then transmit the location of the BCI 1 at intervals so that the location may be tracked by outside devices. Processor 18 may turn off the electrical components of BCI 1 at certain intervals to conserve power, and/or the BCI 1 may be turned on or off remotely.

Alternatively, operations module 16A may have access to the environment outside of BCI 1 such that it can monitor external conditions and/or interact with the outside environment. Operations module 16A may be designed to conduct blood tests, monitor blood pressure or other bodily conditions, house and dispense drugs into the body, and so forth. Information gathered by operations module 16A may then be processed by processor 18 and transmitted to an external receiver via transceiver 22. Thus, as will be understood, an operations module 16A may take various forms and may perform various functions.

FIGS. 3A and 3B illustrate top and bottom cross sections of a top hemisphere 2 and bottom hemisphere 4, as discussed above. As can be seen, bottom hemisphere 4 may also include a transceiver 22 (discussed above), which may also house an RF antenna, for transmitting data out from the BCI 1. As noted above, such transceiver 22 may allow for multiple modules 16A, 16B to be used for purposes other than transmitting data to an outside receiver, and transceiver 22 can be used to control the BCI 1 externally/remotely. Additionally, as shown in FIG. 3A, induction module 10 may include an induction coil such as coil 11.

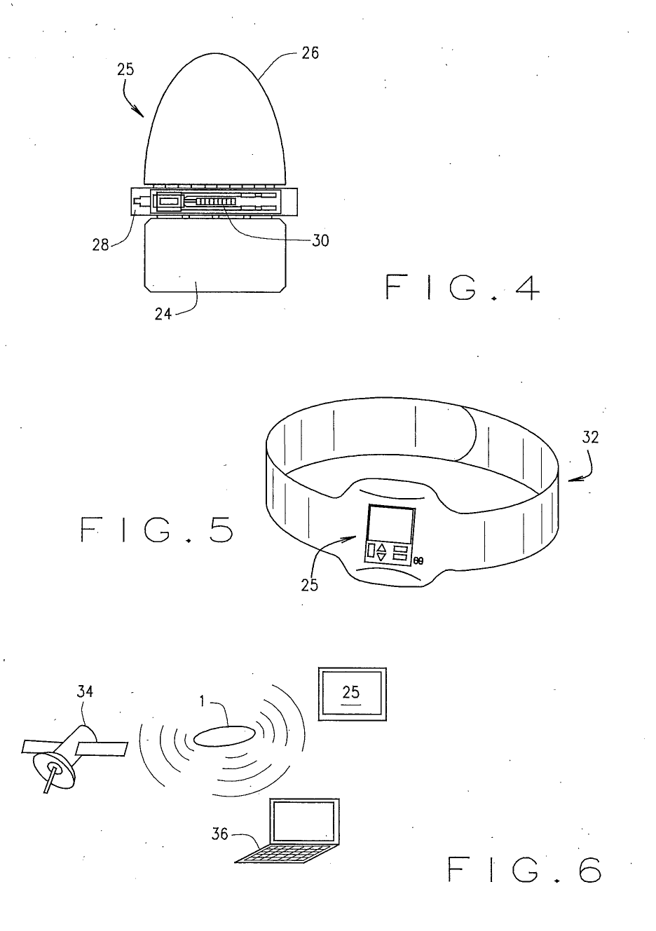

FIG. 4 illustrates a recharging and communication apparatus 25 which includes a power source 24 (e.g., a battery), an induction module 26, a transceiver 28 and a processor 30. As will be understood, power is provided from power source 24, which may be a battery or which may be a device which interfaces with a standard wall electrical outlet, to induction module 26. Induction module 26 includes an induction coil (not shown) as would be understood by one of ordinary skill in the art. This flow of electricity may be controlled by processor 30, such that induction is caused between induction module 26 of the recharging and communication apparatus 25 and the induction module 10 in BCI 1, so as to charge batter 6 in BCI 1. Additionally, processor 30 may control communications sent and may process communications received via transceiver 28. Such transmissions may include instructions to processor 18 of BCI 1, or data sent by processor 18 of BCI 1.

In operation, recharging and communication module 25 is preferably turned on, such that processor 30 causes induction module 26 to create a time-varying or alternating electromagnetic field by appropriately passing electricity through the induction coil therein. When the recharging and communication module 25 is placed close enough to a BCI such as BCI 1, a current is generated in the induction coil of induction module 10 in BCI 1, and this current can be used to charge or recharge battery 6. Data can also be transmitted to (and from) recharging and communication module 25 from (and to) BCI 1 via transceiver 16B and/or antenna 22 in BCI 1, and transceiver 28 in the recharging and communications module 25.

As shown in FIG. 5, in one embodiment recharging and communication module 25 may be installed in a wearable, removable/adjustable strap or band 32 which can be worn around the arm or body of a wearer. In this way, the recharging and communication module 25 may be positioned proximate the BCI 1 (disregarding any tissue therebetween) for wireless charging and communication therewith. Recharging and communication module 25 may alternatively be multiple devices. For example, a first device may wirelessly charge BCI 1 via induction, and a second device may communicate via data transmissions therewith.

FIG. 6 illustrates a block diagram of BCI 1 communicating with various devices, such as recharging and communications module 25 (discussed above). Additionally, BCI 1 may communicate with a satellite 34, such as for GPS purposes, as well as with other computer/tablet/phone systems 36.

Thus, there has been shown and described several embodiments of a novel biocompatible implant device and systems for communicating with and recharging the same wirelessly. As is evident from the foregoing description, certain aspects of the present invention are not limited by the particular details of the examples illustrated herein, and it is therefore contemplated that other modifications and applications, or equivalents thereof, will occur to those skilled in the art. The terms “having” and “including” and similar terms as used in the foregoing specification are used in the sense of “optional” or “may include” and not as “required”. Many changes, modifications, variations and other uses and applications of the present invention will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

What is claimed is:

1. A implant device comprising:
a battery;
an induction module electrically connected to the battery for wirelessly charging the battery via induction;
a processor in electrical communication with the battery for drawing power from the battery;
and a housing made of a biocompatible material, said housing containing the battery, induction module and processor for implantation.

2. The implant device of claim 1 further including a transceiver for at least one of transmitting and receiving information wirelessly.

3. The implant device of claim 1 wherein the induction module and battery are positioned within a recess in a top section of the housing, and the processor is housed within a recess in a bottom section of the housing.

4. The implant device of claim 1 further including an operations module electronically connected to the processor.

5. The implant device of claim 4 wherein the operations module performs at least one of GPS tracking, passive identification, drug dispersal, and monitoring of physical conditions.

6. An implant system comprising:
an implant device including:
a battery;
a first induction module electrically connected to the battery for wirelessly charging the battery via induction;
a processor in electrical communication with the battery for drawing power therefrom;
a first transceiver; and
a housing made of a biocompatible material, said housing containing the battery, induction module, processor and transceiver for implantation; and
a charging apparatus including:
a second induction module for inductively electrically communicating with the first induction coil to generate an electrical current in the first induction coil;
a power source for supplying electricity to the second induction coil; and
a communication apparatus including a processor and a second transceiver for wirelessly communicating with the first transceiver.
